(19) United States
(12) Patent Application Publication
(10) Pub. No.: US 2026/0224241 A1
(43) Pub. Date: Aug. 6, 2026

(54) SUPPORTING DISCHARGE DEVICE AND METHOD FOR SURGERY THEREOF

(71) Applicant: Vigeon Technology Co., Ltd, Taipei-City (TW)

(72) Inventors: Shuo-Lun Lai, Taipei City (TW); Patricia Wanping Wu, Taipei City (TW)

(73) Assignee: Vigeon Technology Co., Ltd., Taipei City (TW)

(21) Appl. No.: 19/636,961

(22) Filed: Apr. 2, 2026

Related U.S. Application Data
(63) Continuation of application No. PCT/US2024/044564, filed on Aug. 30, 2024.
(60) Provisional application No. 63/542,775, filed on Oct. 6, 2023.

(51) Int. Cl. A61B 17/3211

(52) U.S. Cl. CPC A61B 17/3211; A61B 2217/002

(57) ABSTRACT

A supporting device for surgery has a discharge device including a first tubular structure and a relief valve module connected with the first tubular structure. The first tubular structure can be used to encapsulate a scalpel device, and the relief valve module can be connected to a second tubular structure allowing the substance generated during a surgery to be removed. The relief valve module has a rotating assembly used to remain stable when the scalpel device rotates to prevent the second tubular structure twine around the scalpel device during the surgery.

### CROSS REFERENCE TO RELATED APPLICATIONS

This application is a continuation application of PCT Application No. PCT/US2024/044564, filed on August 30th, 2024, which claims the benefit of U.S. Provisional Application No. 63/542,775, filed on October 6th, 2023. The contents of these applications are incorporated herein by reference.

### BACKGROUND OF THE INVENTION

### 1. FIELD OF THE INVENTION

The present disclosure relates to a device for surgery, and more particularly, to a supporting discharge device for surgery.

### 2. DESCRIPTION OF THE PRIOR ART

Electrosurgery is one of the most commonly used energy system in laparoscopic surgery to achieve homeostasis (i.e., bleeding control), which has begun in the early 19^{th} century, via using a high-frequency current generator to supply a current to the tip of a blade of the laparoscopic scalpel and transmit high-energy power to a target tissue. Therefore, the target tissue is heated and cauterized efficiently. The power generated by the current will cause body fluids to vibrate and rub, thereby producing high-level thermal energy, which will evaporate the water among the target tissues and cause the tissues to separate or solidify.

Since the high-level energy is emitted from the scalpel to the target tissues in a short time, a large amount of smoke will produced inside the body chamber where it is located. The smoke plumes will easily diffuse and cause in the impairment of the visual acuity. Recently, removing the smoke generated during the conventionally laparoscopic electrosurgery is accomplished by incorporating a discharge device to the laparoscopic scalpel (i.e., first generation of modified laparoscopic electrosurgery).

However, one of the problems encountered in the first generation of modified laparoscopic electrosurgery is that the tubular structure of the discharge device configured for removing substance generated in the laparoscopic electrosurgery process, e.g. smoke, body fluid or biological tissue, may twine around the scalpel when the scalpel rotates.

In view of the foregoing, it is necessary to provide a novel supporting medical device to overcome the drawbacks faced by existing prior art.

### SUMMARY OF THE INVENTION

Other aspects of the present disclosure will be set forth in the description which follows, and in part will be obvious to one of ordinary skill in the art after perusing the following content. One of ordinary skill in the art may also conceive the content thereof from the implementation of the present disclosure. The advantages disclosed herein may be realized and obtained as particularly pointed out in the appended claims.

To solve the aforementioned problems, the present disclosure provides a supporting device for surgery including a discharge device. The discharge device may include a first tubular structure having a first open end and a second open end departed from the first open end; and a relief valve module connected with the first tubular structure. The relief valve module may include a discharge-adjustable flow channel, an elimination controlling valve, and a rotating assembly. The discharge-adjustable flow channel may have a first opening connected to the first tubular structure, a second opening, and a third opening. The elimination controlling valve may be connected to the discharge-adjustable flow channel and used to selectively open or close the second opening of the discharge-adjustable flow channel. The rotating assembly may be connected to the third opening of the discharge-adjustable flow channel and used to rotate relative to the discharge-adjustable flow channel.

The present disclosure further provides a method for surgery, including using the supporting device of the present disclosure mentioned above to perform surgery on a subject in need thereof.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1A is a schematic diagram of a supporting device for surgery according to an embodiment of the present disclosure.

FIG. 1B is a schematic diagram of a supporting device for surgery according to an embodiment of the present disclosure.

FIG. 1C is a schematic diagram of a supporting device for surgery according to an embodiment of the present disclosure.

FIG. 2A is a schematic diagram of the front view of a discharge device according to an embodiment of the present disclosure.

FIG. 2B is a schematic diagram of a relief valve module according to an embodiment of the present disclosure.

FIG. 2C is a schematic diagram of the side view a relief valve module according to an embodiment of the present disclosure.

FIG. 3A is a schematic diagram of the front view of yet another discharge device according to an embodiment of the present disclosure.

FIG. 3B is a schematic diagram of yet another relief valve module according to an embodiment of the present disclosure.

FIG. 3C is a schematic diagram of the side view yet another relief valve module according to an embodiment of the present disclosure.

FIG. 3D is a schematic diagram of yet another relief valve module according to an embodiment of the present disclosure.

### DETAILED DESCRIPTION

The following descriptions of the embodiments illustrate implementations of the present disclosure, and those skilled in the art of the present disclosure can readily understand the advantages and effects of the present disclosure in accordance with the contents herein. However, the embodiments of the present disclosure are not intended to limit the scope of the present disclosure. The present disclosure can be practiced or applied by other alternative embodiments, and every detail included in the present disclosure can be changed or modified in accordance with different aspects and applications without departing from the essentiality of the present disclosure.

The features such as a ratio, structure, and dimension shown in drawings accompanied with the present disclosure are simply used to cooperate with the contents disclosed herein for those skilled in the art to read and understand the present disclosure, rather than to limit the scope of implementation of the present disclosure. Thus, in the case that does not affect the purpose of the present disclosure and the effect brought by the present disclosure, any change in proportional relationships, structural modification, or dimensional adjustment should fall within the scope of the technical contents disclosed herein.

As used herein, “comprising” (and any variant or conjugation thereof, such as “comprise” or “comprises”), “including” (and any variant or conjugation thereof, such as “include” or “includes”), or “having” (and any variant or conjugation thereof, such as “have” or “has”) a specific element, unless otherwise specified, may include other elements such as components, structures, regions, portions, devices, systems, or connection relationships rather than exclude those elements.

The terms “on,” “side,” “front,” and “between,” described herein are simply used to clarify the embodiments of the present disclosure, rather than used to limit the scope of implementation of the present disclosure. Adjustments, interchanges, and alteration of relative positions and relationships thereof should be considered within the scope of implementation of the present disclosure if the technical contents of the present disclosure are not substantially changed.

The terms “first,” “second,” “third,” “fourth,” etc., used herein are simply used to describe or distinguish elements such as components, structures, regions, portions, devices, or systems, rather than used to limit the scope of implementation of the present disclosure or to limit the spatial order of the elements. In addition, unless otherwise specified, the singular forms “a” and “the” used herein also include plural forms, and the terms “or” and “and/or” used herein are interchangeable.

The terms “connect,” “connected,” “connecting,” and “connects” used herein are used to describe a plurality of components connected together directly or indirectly. “Direct connection” is used to describe a plurality of components connected together upon direct contact; and “indirect connection” is used to describe a plurality of components connected together via at least one connecting component. The terms “connect,” “connected,” “connecting,” and “connects” used herein may be accomplished by magnetic attraction, joggling, conjugation, pivotally connection, binding, seaming, stitching, bonding, adhesion, welding, insertion, clamping, attachment, embedding, integrated molding, or any combination thereof. In at least one of the embodiments of the present disclosure, the plurality of components is “detachable” connected, i.e., the plurality of components may be detached and separated after being connected.

The terms “connect,” “connected,” “connecting,” and “connects” used herein comprises “fixed connection/fixedly connected” and “movable connection/movably connected.”

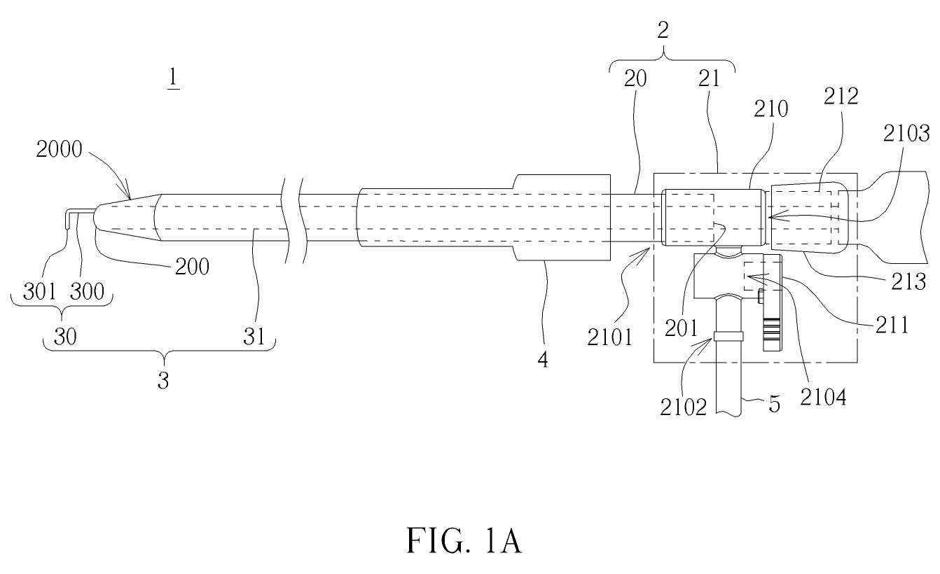

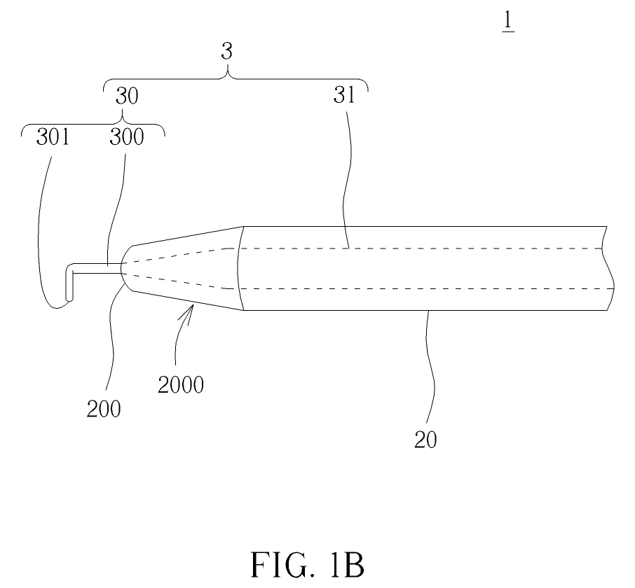

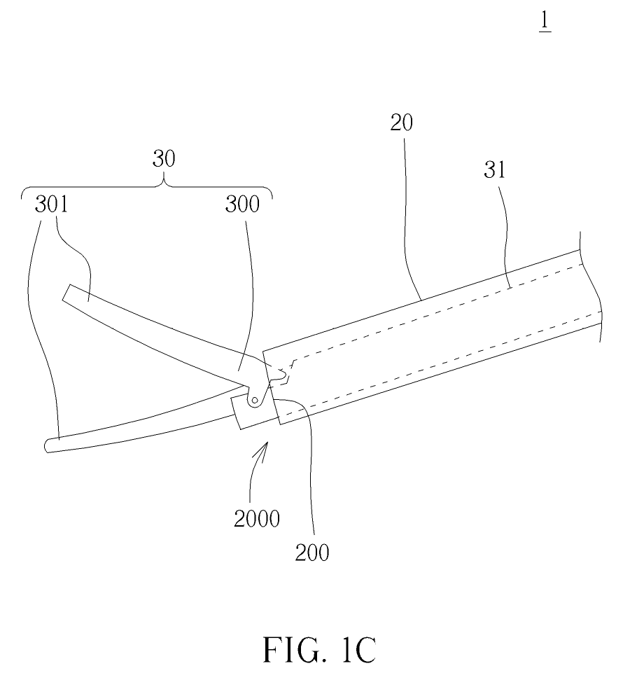

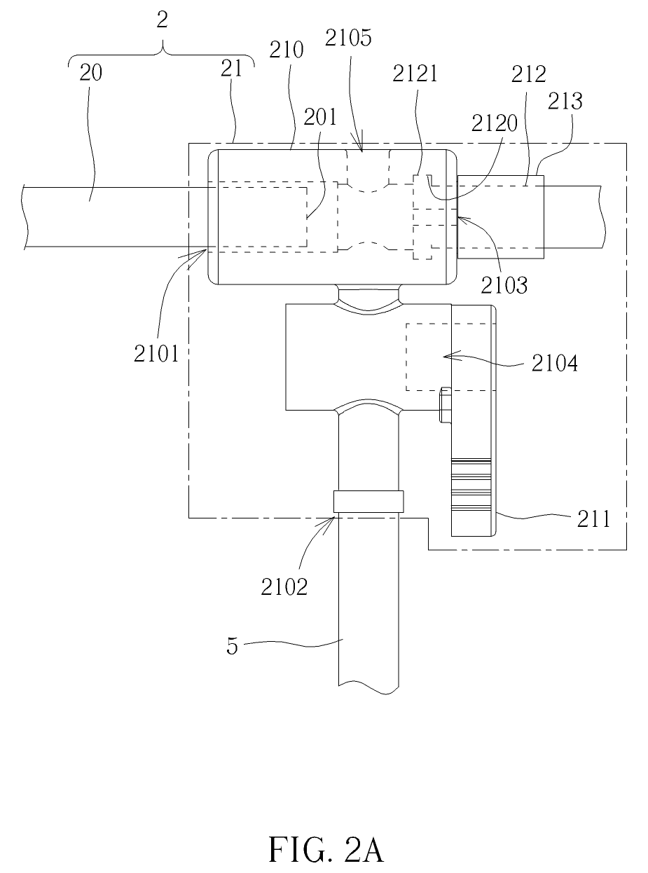

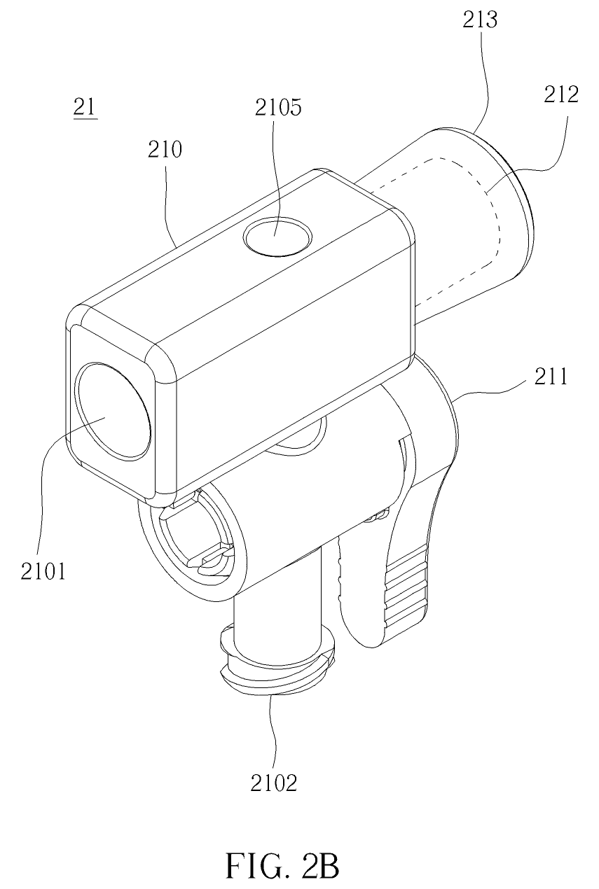

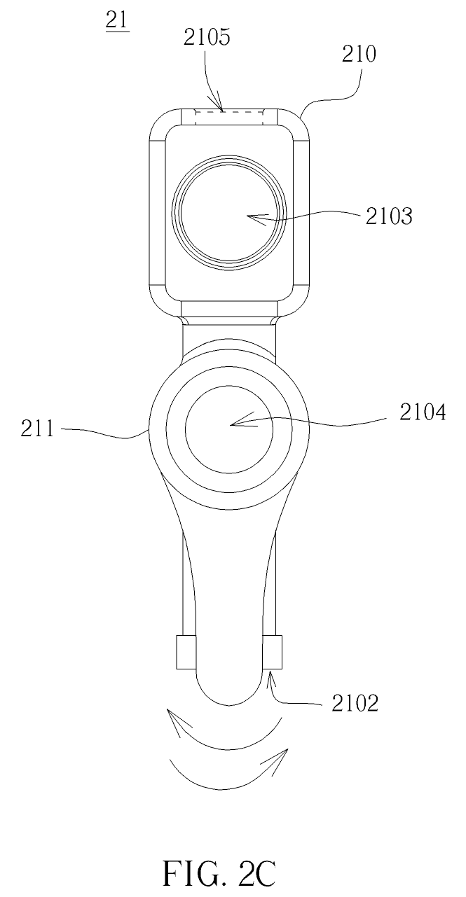

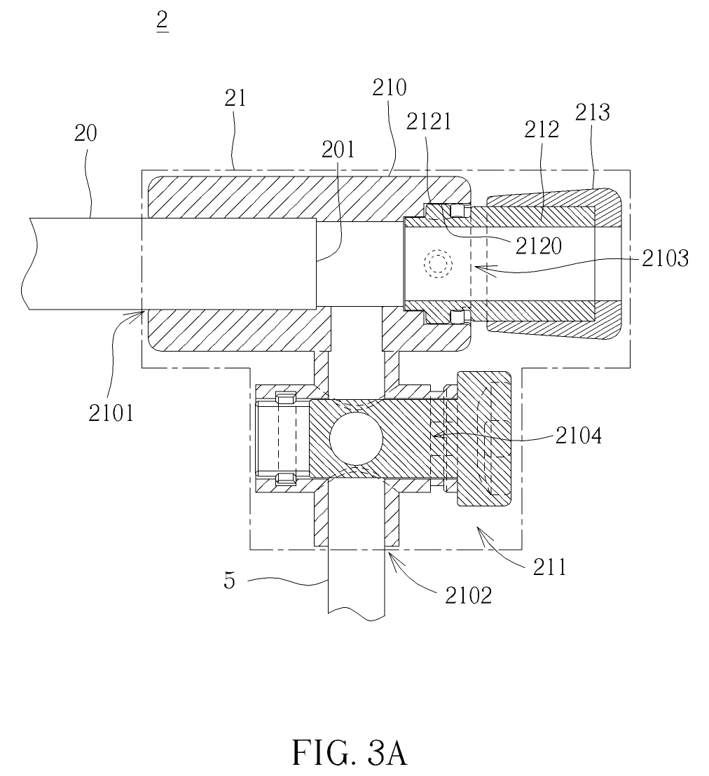

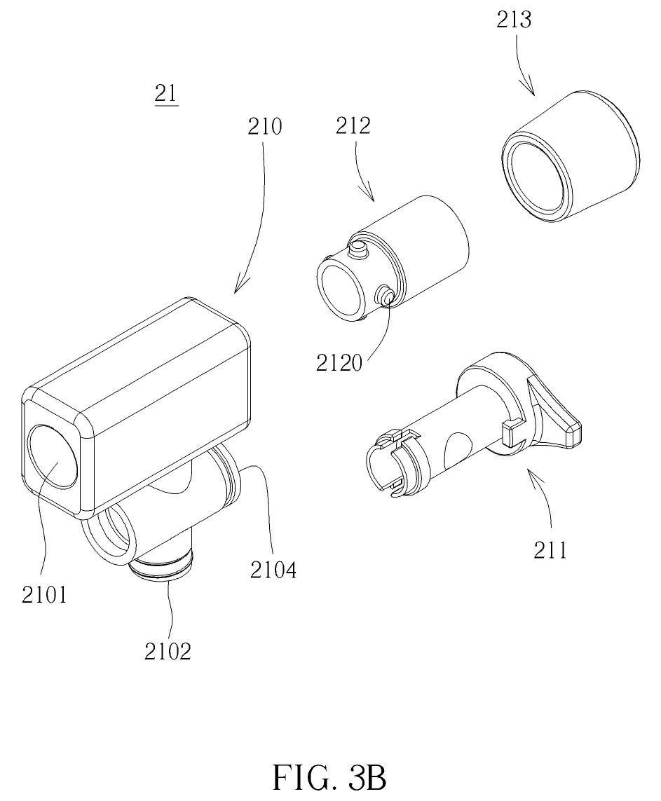

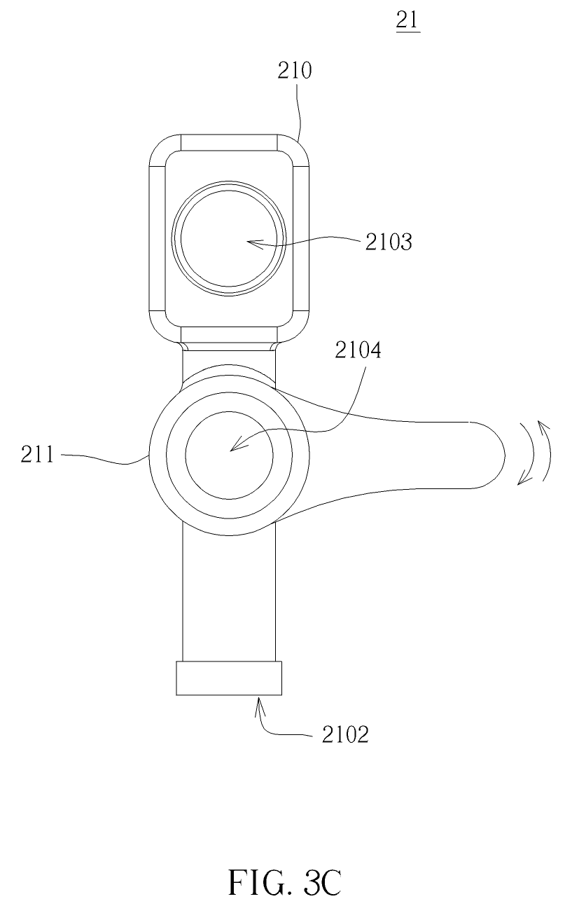

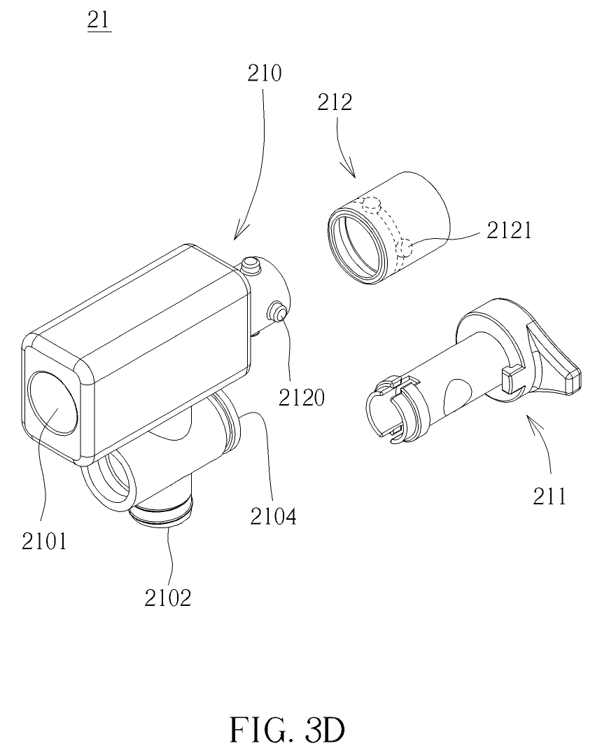

The term “fixed connection/fixedly connected” used herein is used to describe the plurality of components may not be able to move relatively among others after being connected with each other. As shown in FIG. 1A, a discharge device 2 is fixedly connected with a trocar 4; as shown in FIG. 1B, a blade portion 30 of a scalpel device 3 is fixedly connected with a connecting arm 31 of the scalpel device 3; as shown in FIG. 1C, a blade portion 30 of a scalpel device 3 is fixedly connected with a connecting arm 31 of the scalpel device 3; and as shown in FIGS. 2A-3D, a first tubular structure 20 is fixedly connected with a discharge-adjustable flow channel 210 of a relief valve module 21, and a second tubular structure 5 is fixedly connected with the discharge-adjustable flow channel 210 of the relief valve module 21. In one of the embodiments of the present disclosure, as shown in FIGS. 2A and 3A, a rotating assembly 212 with a first convex 2120 (e.g., flange rib) is fixedly connected with a bung 213. In one of the embodiments of the present disclosure, the scalpel device 3 is fixedly connected with the rotating assembly 212 with a first convex 2120 (e.g., flange rib) and the bung 213 (figure not shown). In one of the embodiments of the present disclosure, the scalpel device 3 is fixedly connected with the rotating assembly 212 with a first convex 2120 (e.g., flange ribs) (figure not shown).

The term “movable connection/movably connected” used herein is used to describe the plurality of components may be able to move relatively among others after being connected with each other under a specific scenario. As shown in FIG. 1A, a discharge device 2 is detachable from a scalpel device 3; as shown in FIGS. 1B and 1C, a scalpel device 3 is movable connected with a first tubular structure 20 of the discharge device 2; and as shown in FIGS. 2A-3D, a rotating assembly 212 and the bung 213 are movable connected with the discharge-adjustable flow channel 210 during a surgery; a rotating assembly 212 is movable connected with the discharge-adjustable flow channel 210 during a surgery; and an elimination controlling valve 211 of the relief valve module 21 is movable connected with a discharge-adjustable flow channel 210 of the relief valve module 21 to selectively open or close a second opening 2102 of the discharge-adjustable flow channel 210, moreover, the elimination controlling valve 211 of the relief valve module 21 is capable of controlling the rate of substance discharge via regulating size of the second opening 2102 for fully allowing or restricting the amount of substance passing.

In at least one embodiment of the present disclosure, the relief valve module may further include a bung connected to the rotating assembly.

In at least one embodiment of the present disclosure, the discharge-adjustable flow channel may further include a fourth opening connected to the elimination controlling valve.

In at least one embodiment of the present disclosure, the discharge-adjustable flow channel may further include a fifth opening in communication with the first opening and the second opening. In some embodiments of the present disclosure, the fifth opening may not be communicated with the third opening. In some embodiments of the present disclosure, the fifth opening may or may not be communicated with the fourth opening.

In at least one embodiment of the present disclosure, the rotating assembly may have a first convex portion or a first concave portion; the discharge-adjustable flow channel may have a second convex portion or a second concave portion; and the rotating assembly may be movably connected to the third opening of the discharge-adjustable flow channel via movable connection of the first convex and the second concave portion or movable connection of the first concave portion and the second convex portion. In some embodiments of the present disclosure, the first convex portion or the second convex portion may be, but not limited to, flange ribs.

In at least one embodiment of the present disclosure, the supporting device may further include a second tubular structure connected to the second opening of the discharge-adjustable flow channel.

In at least one embodiment of the present disclosure, the first open end of the first tubular structure may have a head portion.

In at least one embodiment of the present disclosure, the supporting device may further include a trocar, wherein the discharge device may be disposed in the trocar.

In at least one embodiment of the present disclosure, the supporting device may further include a scalpel device having a blade portion and a connecting arm connected to the blade portion. In some embodiments of the present disclosure, the connecting arm of the scalpel device may be at least partially disposed inside the first tubular structure of the discharge device. In some embodiments of the present disclosure, the connecting arm of the scalpel device may be fixedly connected to the rotating assembly of the relief valve module. In some embodiments of the present disclosure, the relief valve module may further include a bung connected to the rotating assembly, and the connecting arm of the scalpel device may be at least partially disposed inside the rotating assembly and the bung of the relief valve module. In some embodiments of the present disclosure, the blade portion of the scalpel device may be at least partially retractable from the first open end of the first tubular structure.

In at least one embodiment of the present disclosure, the first open end of the first tubular structure may be adjacent to or aligned with a bottom of the blade portion of the scalpel device.

In at least one embodiment of the present disclosure, the first tubular structure may be made of a transparent material.

In at least one embodiment of the present disclosure, the relief valve module may be connected to the second open end of the first tubular structure.

In at least one embodiment of the present disclosure, the supporting device may further include a low-pressure evacuation device connected to the second opening of the discharge-adjustable flow channel. In some embodiments of the present disclosure, the low-pressure evacuation device may be enabled by the elimination controlling valve and the fifth opening of the discharge-adjustable flow channel of the relief valve module may remain open, such that the discharge device may be used to operate in a weak discharging mode. In some embodiments of the present disclosure, the low-pressure evacuation device may be enabled by the elimination controlling valve and the fifth opening of the discharge-adjustable flow channel of the discharge device may be closed, such that the discharge device may be used to operate in a strong discharging mode.

In at least one embodiment of the present disclosure, the discharge-adjustable flow channel may be a T-shaped flow channel. In some embodiments, the directions of the openings of the discharge-adjustable flow channel and/or the shape of the discharge-adjustable flow channel can also be modified according to practical needs. For instance, referring to FIG. 2A, the direction of a first opening 2101, second opening 2102, third opening 2103, and/or a fourth opening 2104 may be adjusted.

FIG. 1A shows the supporting device for surgery 1 according to at least one embodiment of the present disclosure, including a discharge device 2, a scalpel device 3, and a trocar 4. The discharge device 2 includes a first tubular structure 20 and a relief valve module 21. The first tubular structure 20 includes a first open end 200 and a second open end 201. The first open end 200 includes a head portion 2000. The relief valve module 21 includes a discharge-adjustable flow channel 210, elimination controlling valve 211, a rotating assembly 212, and a bung 213. The discharge-adjustable flow channel 210 includes a first opening 2101, second opening 2102, third opening 2103, and a fourth opening 2104. The scalpel device 3 includes a blade portion 30 and a connecting arm 31. The blade portion 30 includes a bottom 300 and a tip 301. It should be noted that the quantity and connection of each of the parts are exemplary and can be increased, decreased, or altered according to actual needs. As shown in FIG. 1A, the discharge device 2 is used for allowing a substance generated during a surgical procedure to be removed; the scalpel device 3 is used for cauterizing and removing a biological tissue simultaneously during the surgical procedure; and the trocar 4 is used for creating an opening/port of a subject and allowing the supporting device for surgery 1 to pass through during the surgery procedure.

FIG. 1B shows the supporting device for surgery 1 according to at least one embodiment of the present disclosure, including a first tubular structure 20 and a scalpel device 3. It should be noted that the quantity and connection of each of the parts are exemplary and can be increased, decreased, or altered according to actual needs. As shown in FIG. 1B, the first tubular structure 20 includes a first open end 200 and a head portion 2000, wherein the head portion 2000 may be indented from the surface of the first tubular structure 20, i.e. the head portion 2000 with internal shrinking style. In some embodiment, the first tubular structure 20 is a hollow cylinder, and the first open end 200 is formed at the terminal of the head portion 2000, allowing a substance generated during a surgical procedure to enter the first tubular structure 20 through the first open end 200. As shown in FIG. 1B, the scalpel device 3 includes a blade portion 30 and a connecting arm 31, wherein the blade portion 30 includes a bottom 300 and a tip 301. The type or kind of the scalpel device 3 is not particularly limited in the present disclosure. In some embodiments, the first tubular structure 20 is used to encapsulate at least a portion of, a majority of, or the entire connecting arm 31 of the scalpel device 3. In some embodiments, the first tubular structure 20 is adjacent to or aligned with the bottom 300 of the blade portion 30.

FIG. 1C shows the supporting device for surgery 1 according to at least one embodiment of the present disclosure, including a first tubular structure 20, a blade portion 30, and a connecting arm 31. It should be noted that the quantity and connection of each of the parts are exemplary and can be increased, decreased, or altered according to actual needs. As shown in FIG. 1C, the first tubular structure 20 includes a first open end 200 and a head portion 2000, wherein the head portion 2000 is a blunt end. In some embodiment, user may optionally remove a partial head portion 2000 of the first tubular structure 20 allowing the blade portion 30 and the connecting arm 31 to be disposed in the first tubular structure 20 properly. In some embodiment, the first tubular structure 20 is a hollow cylinder, and the first open end 200 is formed at the terminal of the head portion 2000, allowing a substance generated during a surgical procedure to enter the first tubular structure 20 through the first open end 200. As shown in FIG. 1C, the blade portion 30 includes a bottom 300 and a tip 301. The type or kind of the blade portion 30 and the connecting arm 31 are not particularly limited in the present disclosure. In some embodiments, the first tubular structure 20 is used to encapsulate at least a portion of, a majority of, or the entire connecting arm 31.

In some embodiments, the blade portion 30 of the scalpel device 3 may be in a form or shape of monopolar mode or bipolar mode. In some embodiments, the monopolar mode may be, but not limited to, a hook (e.g., a hook with or without ceramic cone protecting) (FIG. 1B), probe, spatula (e.g., L-shaped, J-shaped or U-shaped), needle, or knife. In some embodiments, the bipolar mode may be, but not limited to, jaws (FIG. 1C), claw, clamp, scissors, shears, clip appliers, or linear cutters.

In some embodiments, the supporting device for surgery 1 may be, but not limited to, a laparoscopic pencil grip, i.e. with a handgrip or a laparoscopic hand instrument, i.e. with a gun or scissor design handle.

### Example 1

FIG. 2A shows the discharge device 2 according to at least one embodiment of the present disclosure, including a first tubular structure 20 and a relief valve module 21; and the second tubular structure 5 according to at least one embodiment of the present disclosure. It should be noted that the quantity and connection of each of the parts are exemplary and can be increased, decreased, or altered according to actual needs. As shown in FIG. 2A, the first tubular structure 20 has a second open end 201 fixedly connected with the relief valve module 21. Furthermore, the relief valve module 21 includes a discharge-adjustable flow channel 210, an elimination controlling valve 211, a rotating assembly 212, and a bung 213. In one embodiment, the discharge-adjustable flow channel 210 includes a first opening 2101, a second opening 2102, a third opening 2103, a fourth opening 2104, and a fifth opening 2105. In one embodiment, the discharge-adjustable flow channel 210 is used for providing a path in direction to remove a substance generated in a surgical process. In one embodiment, the first opening 2101 is connected with the second open end 201 of the first tubular structure 20; the second opening 2102 is connected with the second tubular structure 5, allowing the substance to be removed from the relief valve module 21; the elimination controlling valve 211 is connected with the fourth opening 2104 and used to selectively open or close the second opening 2102, moreover, the elimination controlling valve 211 of the relief valve module 21 is capable of controlling the rate of substance discharge via regulating size of the second opening 2102 for fully allowing or restricting the amount of substance passing; the fifth opening 2105 is used for the operation of a weak or strong discharging mode according to an embodiment of the present disclosure; the third opening 2103 is connected with the rotating assembly 212 via movable connection of the first convex 2120 (e.g., flange ribs) and the second concave portion 2121; and the bung 213 is connected to the rotating assembly 212 as well as the scalpel device 3 of the present disclosure in order to rotate along with the scalpel device 3 of the present disclosure (referring to FIGS. 1A and 1B), allowing the discharge-adjustable flow channel 210 to remain stable during the surgery. In some embodiments, the rotating assembly 212 and the bung 213 may be integrally formed in physical design. In some embodiments, the bung 213 may be optionally removed from the relief valve module 21.

FIGS. 2B and 2C shows the front view and side view of the relief valve module 21, respectively, according to at least one embodiment of the present disclosure, including a discharge-adjustable flow channel 210, a first opening 2101, a second opening 2102, third opening 2103, fourth opening 2104, fifth opening 2105, elimination controlling valve 211, a rotating assembly 212, and a bung 213. It should be noted that the quantity and connection of each of the parts are exemplary and can be increased, decreased, or altered according to actual needs. In one embodiment, the discharge-adjustable flow channel 210 is used for providing a path in direction to remove a substance generated in a surgical process. In one embodiment, the first opening 2101 is connected with the second open end 201 of the first tubular structure 20 (referring to FIG. 2A); the second opening 2102 is connected with the second tubular structure 5 (referring to FIG. 2A), allowing the substance to be removed from the relief valve module 21; the elimination controlling valve 211 is connected with the fourth opening 2104 and used to selectively open or close the second opening 2102, moreover, the elimination controlling valve 211 of the relief valve module 21 is capable of controlling the rate of substance discharge via regulating size of the second opening 2102 for fully allowing or restricting the amount of substance passing; the fifth opening 2105 is used for the operation of a weak or strong discharging mode according to an embodiment of the present disclosure; the third opening 2103 is connected with the rotating assembly 212 via movable connection of the first convex 2120 (e.g., flange ribs) and the second concave portion 2121 (figure not shown); and the bung 213 is connected to the rotating assembly 212 as well as the scalpel device 3 of the present disclosure in order to rotate along with the scalpel device 3 of the present disclosure, allowing the discharge-adjustable flow channel 210 to remain stable during the surgery. In some embodiments, the rotating assembly 212 and the bung 213 may be integrally formed in physical design. In some embodiments, the bung 213 may be optionally removed from the relief valve module 21.

### Example 2

FIG. 3A shows the discharge device 2 according to at least one embodiment of the present disclosure, including a first tubular structure 20 and a relief valve module 21; and the second tubular structure 5 according to at least one embodiment of the present disclosure. It should be noted that the quantity and connection of each of the parts are exemplary and can be increased, decreased, or altered according to actual needs. As shown in FIG. 3A, the first tubular structure 20 has a second open end 201 fixedly connected with the relief valve module 21. Furthermore, the relief valve module 21 includes a discharge-adjustable flow channel 210, an elimination controlling valve 211, a rotating assembly 212, and a bung 213. In one embodiment, the discharge-adjustable flow channel 210 includes a first opening 2101, a second opening 2102, a third opening 2103, and a fourth opening 2104. In one embodiment, the discharge-adjustable flow channel 210 is used for providing a path in direction to remove a substance generated in a surgical process. In one embodiment, the first opening 2101 is connected with the second open end 201 of the first tubular structure 20; the second opening 2102 is connected with the second tubular structure 5, allowing the substance to be removed from the relief valve module 21; the elimination controlling valve 211 is connected with the fourth opening 2104 and used to selectively open or close the second opening 2102, moreover, the elimination controlling valve 211 of the relief valve module 21 is capable of controlling the rate of substance discharge via regulating size of the second opening 2102 for fully allowing or restricting the amount of substance passing; the third opening 2103 is connected with the rotating assembly 212 via movable connection of the first convex 2120 (e.g., flange ribs) and the second concave portion 2121; and the bung 213 is connected to the rotating assembly 212 as well as the scalpel device 3 of the present disclosure in order to rotate along with the scalpel device 3 of the present disclosure (referring to FIGS. 1A and 1B), allowing the discharge-adjustable flow channel 210 to remain stable during the surgery. In some embodiments, the rotating assembly 212 and the bung 213 may be integrally formed in physical design. In some embodiments, the bung 213 may be optionally removed from the relief valve module 21.

FIGS. 3B and 3C shows the front view and side view of the relief valve module 21, respectively, according to at least one embodiment of the present disclosure, including a discharge-adjustable flow channel 210, a first opening 2101, a second opening 2102, third opening 2103, a fourth opening 2104, an elimination controlling valve 211, a rotating assembly 212 with a first convex 2120, and a bung 213. It should be noted that the quantity and connection of each of the parts are exemplary and can be increased, decreased, or altered according to actual needs. In one embodiment, the discharge-adjustable flow channel 210 is used for providing a path in direction to remove a substance generated in a surgical process. In one embodiment, the first opening 2101 is connected with the second open end 201 of the first tubular structure 20 (referring to FIG. 3A); the second opening 2102 is connected with the second tubular structure 5 (referring to FIG. 3A), allowing the substance to be removed from the relief valve module 21; the elimination controlling valve 211 is connected with the fourth opening 2104 and used to selectively open or close the second opening 2102, moreover, the elimination controlling valve 211 of the relief valve module 21 is capable of controlling the rate of substance discharge via regulating size of the second opening 2102 for fully allowing or restricting the amount of substance passing; the third opening 2103 is connected with the rotating assembly 212 via movable connection of the first convex 2120 (e.g., flange ribs) and the second concave portion 2121 (figure not shown); and the bung 213 is connected to the rotating assembly 212 with the first convex 2120 (e.g., flange ribs) as well as the scalpel device 3 of the present disclosure (referring to FIGS. 1A and 1B) in order to rotate along with the scalpel device 3 of the present disclosure (referring to FIGS. 1A and 1B), allowing the discharge-adjustable flow channel 210 to remain stable during the surgery. In some embodiments, the rotating assembly 212 with the first convex 2120 (e.g., flange ribs) and the bung 213 may be integrally formed in physical design. In some embodiments, the bung 213 may be optionally removed from the relief valve module 21.

FIG. 3D shows the front view of the relief valve module 21 according to at least one embodiment of the present disclosure, including a discharge-adjustable flow channel 210 with a second convex portion 2120 (e.g., flange ribs), a first opening 2101, a second opening 2102, a fourth opening 2104, elimination controlling valve 211, and a rotating assembly 212. It should be noted that the quantity and connection of each of the parts are exemplary and can be increased, decreased, or altered according to actual needs. In one embodiment, the discharge-adjustable flow channel 210 with the second convex portion 2120 (e.g., flange ribs) is used for providing a path in direction to remove a substance generated in a surgical process. In one embodiment, the first opening 2101 is connected with the second open end 201 of the first tubular structure 20 (referring to FIG. 3A); the second opening 2102 is connected with the second tubular structure 5 (referring to FIG. 3A), allowing the substance to be removed from the relief valve module 21; the elimination controlling valve 211 is connected with the fourth opening 2104 and used to selectively open or close the second opening 2102, moreover, the elimination controlling valve 211 of the relief valve module 21 is capable of controlling the rate of substance discharge via regulating size of the second opening 2102 for fully allowing or restricting the amount of substance passing; the third opening 2103 is connected with the rotating assembly 212 via movable connection of the first concave portion 2121 and the second convex portion 2120; and the rotating assembly 212 is connected with the scalpel device 3 as well as the second convex portion 2120 of the discharge-adjustable flow channel 210 in order to rotate along with the scalpel device 3 of the present disclosure (referring to FIGS. 1A and 3A), allowing the discharge-adjustable flow channel 210 to remain stable during the surgery. In some embodiments, the rotating assembly 212 has the first concave portion 2121 corresponding to the second convex portion 2120 of the discharge-adjustable flow channel 210, and the first concave portion 2121 are formed internally in the rotating assembly 212. In some embodiments, the rotating assembly 212 is connected to the second convex 2120 of the discharge-adjustable flow channel 210 via the aforementioned the first concave portion 2121.

The foregoing outlines the features of several embodiments, enabling those skilled in the art to fully appreciate the aspects of the present disclosure. Those skilled in the art should recognize that the present disclosure provides a foundation for designing or modifying other processes and structures to achieve substantially the same functions and/or substantially the same results as those of the embodiments introduced herein. Furthermore, such equivalent arrangements do not deviate from the spirit and scope of the present disclosure, and various changes, substitutions, and alterations may be made without so departing.

What is claimed is:

1. A supporting device for surgery, comprising:
a discharge device comprising:
a first tubular structure having a first open end and a second open end departed from the first open end; and
a relief valve module connected with the first tubular structure, comprising:
a discharge-adjustable flow channel having a first opening connected to the first tubular structure, a second opening, and a third opening;
an elimination controlling valve connected to the discharge-adjustable flow channel and configured to selectively open or close the second opening of the discharge-adjustable flow channel; and
a rotating assembly connected to the third opening of the discharge-adjustable flow channel and configured to rotate relative to the discharge-adjustable flow channel.

2. The supporting device of claim 1, wherein the relief valve module further comprises a bung connected to the rotating assembly.

3. The supporting device of claim 1, wherein the discharge-adjustable flow channel further comprises a fourth opening connected to the elimination controlling valve.

4. The supporting device of claim 1, wherein the discharge-adjustable flow channel further comprises a fifth opening in communication with the first opening and the second opening.

5. The supporting device of claim 1, wherein
the rotating assembly has a first convex portion or a first concave portion;
the discharge-adjustable flow channel has a second convex portion or a second concave portion; and
the rotating assembly is movably connected to the third opening of the discharge-adjustable flow channel via movable connection of the first convex and the second concave portion or movable connection of the first concave portion and the second convex portion.

6. The supporting device of claim 1, further comprising a second tubular structure connected to the second opening of the discharge-adjustable flow channel.

7. The supporting device of claim 1, further comprising a trocar, wherein the discharge device is disposed in the trocar.

8. The supporting device of claim 1, further comprising a scalpel device comprising a blade portion and a connecting arm connected to the blade portion.

9. The supporting device of claim 8, wherein the connecting arm of the scalpel device is at least partially disposed inside the first tubular structure of the discharge device.

10. The supporting device of claim 8, wherein the connecting arm of the scalpel device is fixedly connected to the rotating assembly of the relief valve module.

11. The supporting device of claim 10, wherein the relief valve module further comprises a bung connected to the rotating assembly, and the connecting arm of the scalpel device is at least partially disposed inside the rotating assembly and the bung of the relief valve module.

12. The supporting device of claim 1, wherein the first open end of the first tubular structure is adjacent to or aligned with a bottom of the blade portion of the scalpel device.

13. The supporting device of claim 8, wherein the blade portion of the scalpel device is at least partially retractable from the first open end of the first tubular structure.

14. The supporting device of claim 1, wherein the first tubular structure is made of a transparent material.

15. The supporting device of claim 1, wherein the relief valve module is connected to the second open end of the first tubular structure.

16. The supporting device of claim 1, further comprising a low-pressure evacuation device connected to the second opening of the discharge-adjustable flow channel.

17. The supporting device of claim 16, wherein the discharge-adjustable flow channel further comprises a fifth opening in communication with the first opening and the second opening, and the low-pressure evacuation device is enabled by the elimination controlling valve and the fifth opening of the discharge-adjustable flow channel of the relief valve module remains open, such that the discharge device is configured to operate in a weak discharging mode.

18. The supporting device of claim 16, wherein the discharge-adjustable flow channel further comprises a fifth opening in communication with the first opening and the second opening, and the low-pressure evacuation device is enabled by the elimination controlling valve and the fifth opening of the discharge-adjustable flow channel of the discharge device is closed, such that the discharge device is configured to operate in a strong discharging mode.

19. The supporting device of claim 1, wherein the discharge-adjustable flow channel is a T-shaped flow channel.

20. A method for surgery, comprising using the supporting device of claim 1 to perform surgery on a subject in need thereof.
